(19) United States
(12) Patent Application Publication
(10) Pub. No.: US 2013/0010259 A1
(43) Pub. Date: Jan. 10, 2013

(54) Region based vision tracking system for imaging of the eye for use in optical coherence tomography

(75) Inventor: Matthew Carnevale, Medford, MA (US)

(73) Assignee: Escalon Digital Vision, Inc.

(21) Appl. No.: 13/135,419

(22) Filed: Jul. 5, 2011

(51) Int. Cl. A61B 3/113; A61B 3/14

(52) U.S. Cl. 351/206; 351/209; 351/246

(57) ABSTRACT

For optical coherence tomography engines a method for eliminating the effects of the movement of the eye on the optical coherence tomography scan calculates the motion of the eye from an image from an auxiliary scanning system and compares a reference region to a corresponding region in the image associated with the next frame, with the change in position sensing the motion of the eye. This is followed by utilizing this sensed motion to generate accurate offsets for the scanning mirror patterns of the OCT engine. Additionally, scan skipping is utilized to obviate the effects of rapid eye movement that occur at rates faster than the image acquisition rate.

### FIELD OF THE INVENTION

This invention relates to optical coherence technology and more particularly to region-based image tracking for eliminating the effects of the movement of the eye on the optical coherence tomography scan.

### BACKGROUND OF THE INVENTION

Optical coherence tomography (OCT) is a technology for performing high resolution cross sectional imaging that can provide images of tissue structure on the micron scale in vivo and in realtime. OCT is a method of interferometry that uses light containing a range of optical frequencies to determine the scattering profile of a sample. Optical coherence tomography as a tool for evaluating biological materials was first disclosed in the early 1990s and is described in U.S. Pat. No. 5,321,501 in which the optical coherence tomography was used for fundus imaging. While there are various systems for obtaining time domain cross sectional images of the retina, in recent years it has been demonstrated that frequency domain OCT has significant advantages in speed and signal-to-noise ratios compared to time domain OCT.

In frequency domain OCT a light source capable of emitting a range of optical frequencies excites an interferometer. The interferometer combines the light returned from a sample with a reference beam of light from the same source. The light to and

### FIELD OF THE INVENTION

This invention relates to optical coherence technology and more particularly to region-based image tracking for eliminating the effects of the movement of the eye on the optical coherence tomography scan.

### BACKGROUND OF THE INVENTION

Optical coherence tomography (OCT) is a technology for performing high resolution cross sectional imaging that can provide images of tissue structure on the micron scale in vivo and in realtime. OCT is a method of interferometry that uses light containing a range of optical frequencies to determine the scattering profile of a sample. Optical coherence tomography as a tool for evaluating biological materials was first disclosed in the early 1990s and is described in U.S. Pat. No. 5,321,501 in which the optical coherence tomography was used for fundus imaging. While there are various systems for obtaining time domain cross sectional images of the retina, in recent years it has been demonstrated that frequency domain OCT has significant advantages in speed and signal-to-noise ratios compared to time domain OCT.

In frequency domain OCT a light source capable of emitting a range of optical frequencies excites an interferometer. The interferometer combines the light returned from a sample with a reference beam of light from the same source. The light to and from the sample is aimed via a scanning mechanism, which can be a single mirror, or a series of mirrors, such as a pair for steering the beam in the X and Y dimensions to create a scan pattern. The intensity of the combined light from the sample and reference arms is recorded as a function of optical frequency to form an interference spectrum. A Fourier transform of the interference spectrum provides the reflectance distribution along the depth at a point on the sample with a number side by side depth distributions resulting in a scan of the thickness of the retina along the scan line.

Certain difficulties have arisen in existing OCT systems including for instance eye movement during the measurement period which is said to cause a wide variety of difficulties. It is said that efforts have been made to increase the speed of data collection to reduce the effects of motion of the eye, and also it is said that various approaches have been suggested to measure eye motion and then compensate for the motion.

In short, a major problem in terms of resolution of the scanned area is that the scanning takes a certain amount of time in which the eye must not move. However, involuntary motions of the eye always exist which cause the data not to line up properly and it is difficult to correlate the scans with the image of the retina, absent eye motion tracking.

It is noted that non-tracking systems might try to image the retina out anywhere from half a second to perhaps a full second. However the artifacts obtained with such a full second scanning period can be excessive in terms of eye motion, whereas half a second corresponds to a comfort zone where one can image without picking up too many motion effects. Note in terms of registration problems, in many OCT scanning systems there is a secondary image from an auxiliary imager, such as a camera which shows the retina in terms of the visible tissue in the eye. One then tries to correlate the particular depth in a scan to a particular visible region as viewed in the secondary imaging system.

If there is motion of the eye during a scan, many issues can arise such as blurred or skewed images with large portions of the data twisted, crooked and empty portions of missing data. Additionally, instead of obtaining a perfect picture of the image, one will have misalignment of features and one will not be able to line up the scans to the secondary image of the retina. For instance if one has a very high resolution raster scan of several thousand points, if half way through the scan an involuntary motion of the eye is encountered, then the eye will slightly rotate and look off in a slightly different direction. When the system continues to scan, the point of impact of the spot of light on the retina is shifted so that one is basically not looking at the appropriate portion of the retina. The result is that several hundred or even thousands of scan points do not correlate with the secondary retinal image.

Motion compensation is described in US Patent Publication No. US 2003/0160943, published Aug. 28, 2003 which was filed Jul. 26, 2002 and is a result of a Continuation-in-Part of application Ser. No. 10/086,092 filed on Feb. 26, 2002.

In this patent publication eye movement is detected through measuring the intensity of light reflected back from a reference feature, with the intensity of the reflected light indicating eye motion. The change in the intensity of the return light based on eye motion is not a particularly accurate means of tracking eye motion and there is a requirement for a less complex, more economical and more accurate method of compensating for eye motion in optical coherence tomography.

As described in U.S. Pat. No. 7,805,009 “A Method of Measuring Motion Using a Series of Partial Images from an Imaging System”, a line scan image is used to determine eye movement by comparing a line of image data with a reference image to detect displacement. However, in this patent only a line of data is analyzed. While this line scanning technique speeds motion detection because only a line at a time is analyzed, there are accuracy problems. Particularly, no image analysis is made of an entire region which would more accurately characterize eye movement. Instead this patent teaches line-at-a-time analysis.

It will therefore be appreciated that an image line is analyzed as it is coming in. Thus, the system is not actually collecting the whole image and then analyzing a region within it to fit to a reference image and develop a correlated fit. In this patent one does not collect all of the information from the imager so that one can obtain a more detailed fit. As a result one is not taking advantage of all of the information that is available from the entire image.

While the line analysis motion detector is used to optimize how quickly the system can characterize the motion of the eye, there are two potential loop holes. First, regardless of the line analysis one cannot process the data fast enough to generate scan pattern offsets. Secondly, even if one can generate the offsets, there is not enough information to effectively cancel out the eye motion. Therefore a method is required to perform a more accurate scan by using all of the information available in an image and to perform the scan in a manner that will permit effective eye movement cancellation.

The problem with using a line of data is that the OCT scan may be built upon inaccurate data. For instance, noise or vignetting artifacts at the ends of a line, as well as small vessel movements from the pulse of blood being pumped from the heart may affect measurement of retinal displacement during eye movement. There needs to be some way to eliminate these artifacts from the data or ignore inaccurate data. If not, the scan image will not be as sharp as it could be and the features the doctor wishes to see may be blurred or not properly registered.

There is therefore a requirement for an inexpensive and accurate motion tracking system which can offset the scan pattern to be able to take out the effects of eye motion or to skip frames in which too much motion is sensed.

### SUMMARY OF INVENTION

In the subject invention an image of the eye is captured and a reference tracking region on the retina is recorded. This is done within one frame. The subsequent frame generates an image of the region which all of the pixels in the image are compared with the pixels in image of the region from the first frame and motion is detected in terms of a displacement of the second region with respect to the first region. The deflection distance is converted into signals which drive the X and Y scan mirror patterns to offset them to cancel out the motion. In one embodiment, during a scan any motion up to 1/30^{th} of a second is canceled by offsetting the scan mirror patterns directing the scanning operation.

Unlike the above single line comparisons, in the subject system, while line scans are used to build the full frame, the entire frame is collected and a data rich two dimensional region is registered against a reference region to derive offsets. While the subject system is slower than the line analysis systems, this system functions despite slow eye motion. For rapid eye motion these rapid motions are ignored and the system waits until these rapid motions pass by a process called scan skipping.

As a result the subject system takes into account all of the data rich information available to provide a complete image analysis on an entire region as opposed to a line. The result is superior measurement accuracy and repeatability.

Moreover, in the subject case vascular-based feature extraction is performed over the region and this feature extraction permits more accurate registrations and thus is capable of single pixel and even sub-pixel accuracies.

In one embodiment, a secondary imager outputs a view of the retina at 30 frames per second, in which any change in the reference tracking region is measured in terms of the displacement of the region when going from one frame to another.

In order to obtain the frame to frame displacement of the reference tracking region, in one embodiment image analysis software is utilized to determine the offset between the two regions. One such region analysis system is described in a paper entitled The Dual Bootstrap Iterative Closest Point Algorithm with Application to Retinal Image Registration by Charles V. Stewart et al., the IEEE International Symposium on Biomedical Imaging, July 2002.

Moreover, in one embodiment, offsetting of the OCT scan mirror patterns involves generating a matrix that describes interframe motion, with the matrix being used to compute the scan mirror drive signals to offset the scanner mirror patterns in a way to cancel the effect of the motion. The matrix is also useful to correct for viewpoint shifts and rotational skewing.

Thus, as the scanner rotates the scan mirrors in a pattern to create the OCT scan, the matrix computation is invoked to offset the scanning mirror patterns by values calculated from the matrix. During a scan the scan mirror controller calculates where the next scan point is in the pattern, with the matrix values being applied to make sure that the next scan point is appropriately offset such that the same position on the retina is scanned, despite the fact that the retina may have moved due to eye movement. Note that the matrix generated offsets are applied to existing firmware that controls the scanning mirror axes.

In another embodiment, scan skipping is employed to obviate the effects of rapid eye movements which occur at rates faster than the image acquisition rate. Here an eye motion threshold is applied during region tracking. If the movement is less than or greater than the threshold the offsets generated are applied to the scanner mirror patterns as a matter of course. If the threshold is exceeded, all data collected between frames since the last frame below the threshold is ignored and keeps being ignored until the eye movement settles down again below the threshold.

Thus, one is only collecting data when there is no eye motion. The OCT scan position is reset to the last known scan location prior to exceeding the threshold. This in turn results in a better image presented to the doctor because there are no gaps or alignment errors in the data.

As will be appreciated, it is the purpose of the subject motion capture invention to take two dimensional region-based algorithms and calculate motion by registering all of the data rich information from a region in one image with a corresponding region in a next adjacent image. This is followed by utilizing the resulting transformation matrix to generate offset values in the X and Y directions, with the values used to offset the scanning mirror patterns in the OCT scanner.

While the subject invention has been described in terms of retinal imaging, it is also possible to image the front or anterior segment of the eye in the same manner, although the landmarks and features used for registration will be different. Thus, it is possible to get OCT scans of the thickness of an anterior segment of the eye as opposed to a posterior segment.

In this regard, in determining the position of the eye one can lock onto the pattern of the iris, sclera or pupil as opposed to various vascular features on the retina. This iris pattern movement can in turn be utilized in generating a transformation matrix to characterize motion of the eye. In addition, a reflection of an external object on the optical surfaces of the eye, known as Purkinje images, can also be tracked to determine motion.

In the case of anterior segment OCT, the region imaged is much larger than that of the retina, and the structures are more intricate requiring deeper penetration and a longer depth profile. Because of this, there is a need to not only scan the appropriate point, but also to align the vectors of the OCT depth profile in 3D space to ensure the anatomy imaged is properly registered in all dimensions, X, Y and Z. The translation matrix obtained from the image processing algorithms can also be used to perform this registration after the data is acquired. This type of alignment can also be applied to OCT datasets of very large retinal areas.

Note, in the subject invention one correlates the depth scan to the view of the retina that has been previously obtained. Another benefit is that in follow-up scanning if one wants to scan the exact same location one can utilize the same image displacement software to correct scan line positioning so that the second scanning operation can be made coincident with the first scanning operation. This makes it possible to detect the differences in tissue thickness at the same spot at two different times so that one can see disease progression.

In summary, for optical coherence tomography engines a method for eliminating the effects of the movement of the eye on the optical coherence tomography scan calculates the motion of the eye from an image from an auxiliary scanning system and compares a reference region to a corresponding region in the image associated with the next frame, with the change in position sensing the motion of the eye. This is followed by utilizing this sensed motion to generate accurate offsets for the scanning mirror patterns of the OCT engine. Additionally, scan skipping is utilized to obviate the effects of rapid eye movement that occur at rates faster than the image acquisition rate.

### BRIEF DESCRIPTION OF THE DRAWINGS

These and other features of the subject invention will be better understood in connection with the Detailed Description, in conjunction with the Drawings, of which:

FIG. 1 is a diagrammatic illustration of a view of the retina of an eye which has moved such that there is a displacement between a data rich region of an image of the retina including a large number of reference tracking features highlighted by image processing and filtering techniques and a subsequently obtained image of the region to ascertain displacement of the region due to eye movement; and,

FIG. 2 is a block diagram of the subject system illustrating the capturing of the region of FIG. 1 on an auxiliary imager and the utilization of the displacement of this data rich region, frame by frame, to calculate the magnitude and direction of the eye motion, to generate a matrix corresponding to the magnitude and direction of the eye motion and to generate scan mirror control signals based on matrix values that are coupled to the scan mirror controller to offset the scan mirror patterns to cancel the effects of eye motion, with a scan skipping subsystem employed to inhibit data collection when eye motion exceeds a predetermined threshold, thus to act on the detection of slow or minor eye movements, while waiting to collect data until eye motion has quieted down.

### DETAILED DESCRIPTION

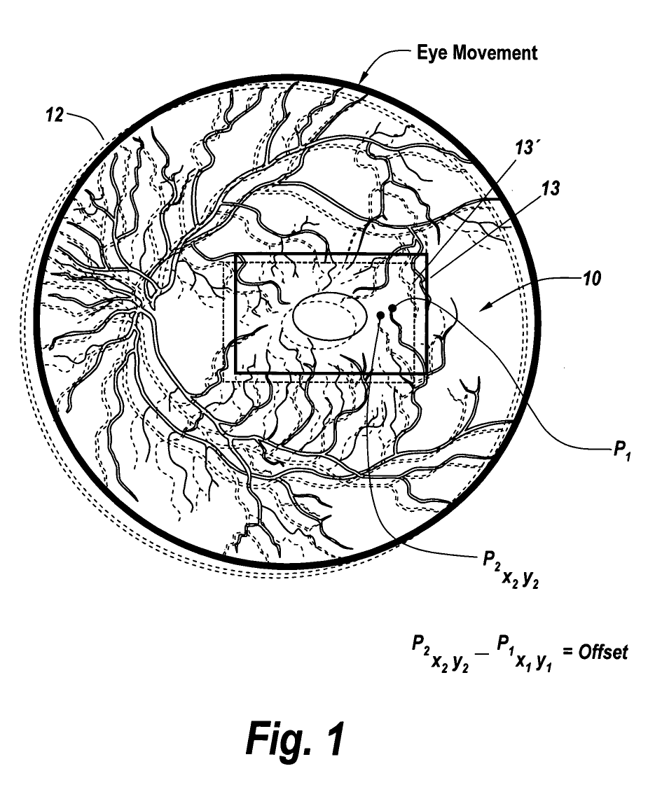

Referring now to FIG. 1, an image 10 of the retina of an eye is illustrated having a data rich region of features including vessels and other artifacts clearly visible on the surface of the retina. Eye movement is shown in dotted line in which the eye moves as illustrated by dotted line 12 resulting in movement of the data rich region image of the retina as viewed by an auxiliary imager.

Here it can be seen a point in a data rich region 13 which constitutes for instance a reference tracking feature moves from point P₁ at X₁ Y₁ to P₂ at X₂ Y₂. This corresponds to a shift in region 13 as shown by dotted outline 13′. Note that region 13 of the retina sensed includes virtually hundreds and even thousands of these points, the movement of which is calculated through comparing a registration process so as to take advantage of all of the information that is available in the region. This is contrasted to the use of a single line which carries with it a number of artifacts that corrupt the data collected.

In one embodiment the data rich region is processed using methods from the The Dual Bootstrap Iterative Closest Point Algorithm with Application to Retinal Image Registration system described in the aforementioned article by Stewart el al. The Stewart el al system is used to develop a transformation matrix describing the movement of the region based on the detected vasculature of the retina. This vasculature can be extracted with feature extraction techniques described in this article so that registration to a reference region results in a significant accuracy increase over the aforementioned line scanning. As a result single pixel and even sub pixel accuracies can be achieved in the positioning afforded by the OCT scanning mirror system. The resultant A-scans or B-Scans present to the doctor much improved resolution and much improved registration to permit accurate diagnosis and delivery of therapeutic modalities.

While one registration technique is described herein, any registration or cross correlation technique which takes advantage of all of the information in a region of the image is within the scope of this invention.

As will be described, the registration of an entire image requires waiting for all of the line scans of an image to be captured and thus can take more time than is desired to be able to capture rapid eye movement. It will be noted that although the aforementioned line correlation techniques were designed to speed up motion detection to capture fast eye movement, such techniques were found to introduce artifacts in the collected data to such an extent that resultant A-scans alignment was not reliable.

To counter the problem of latency from processing entire regions, in the subject invention the magnitude of the detected eye motion is sensed. If the eye motion is slow enough to be canceled by region based processing, then the scan mirror patterns are offset in accordance with the region to region offset matrix obtained from the displacement of the entire region.

However if the sensed motion exceeds a predetermined threshold, then the data collected is ignored and the process is rewound to the last good registration once the sensed eye motion calms down to an acceptable level. This leaves the collected data set uncorrupted. The result is a marked increase in resolution and registration accuracies.

Thus, it will be appreciated that the subject compensation system operates on sequential image frames, with the movement of a region in the image from one frame to another providing the sensed parameter by which scanning mirror patterns of the OCT scanner can be offset. This is unlike measuring reflectance intensity or phase, or any other parameter for offsetting OCT scanning mirror patterns.

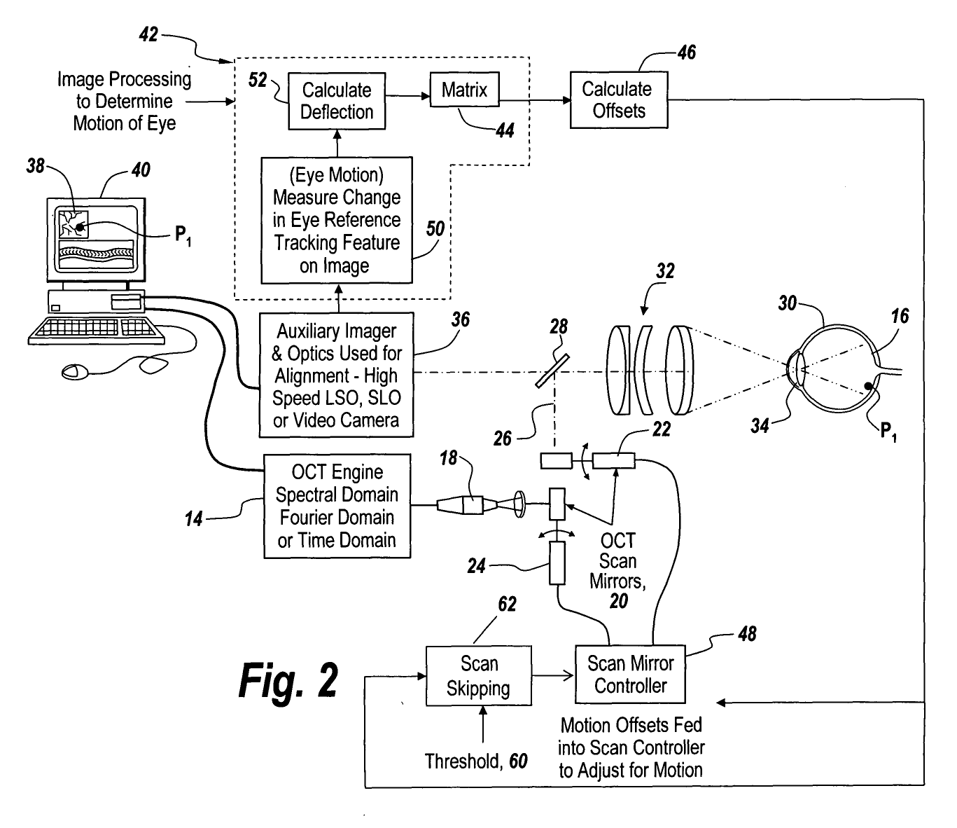

Referring now to FIG. 2, the subject system operates on an OCT engine 14 that scans the retina 16 of eye 30 by projecting a light beam from optics 18 to OCT scan mirrors 20 driven by actuators 22 and 24 in orthogonal directions. This moves beam 26 which is redirected through a beam splitter 28 and through optics 32 such that the beam passes through the cornea 34 of eye 30 and onto the retina.

What is described above is called the primary path, whereas an auxiliary imager 36 is aligned along the primary optical path and provides an image of the surface of retina 16 which may be displayed as an image 38 on a computer monitor 40, with the displayed image displaying the vascularization within the sensed region.

The auxiliary imager and optics are generally used for alignment of the start position for OCT scans and are typically video cameras, line scanning ophthalmoscopes (LSO) or scanning laser ophthalmoscopes (SLO). An SLO is similar to an LSO, but utilizes a single point detector that is scanned over the eye in a raster pattern to create lines that are stacked up to create a two dimensional image.

Alternatively, the auxiliary imager can also use the optical coherence beam itself. The surface of the eye can be extracted from the OCT scan, thus generating an en face or forward face view of the eye equivalent to that of the above-mentioned auxiliary imagers.

This scanning is utilized solely to build up a line scanned image of the surface of the retina and is not the same as the OCT scanning.

The output of the auxiliary imager optics is an image which is stored and is provided to an image processing module 42 that determines through detection of the change of the data rich region position from one frame to the next the change in position used to calculate a transformation matrix 44. This matrix is then used to calculate offsets at drive 46 which are converted to a series of signals that are applied to a scan mirror controller 48 to offset the scan mirror patterns during the scanning process associated with the OCT scan.

These offsets are such that the point of impingement of beam 26 on a retina 16 remains fixed on the same point even when the eye moves. This is because the beam will be moved to the exact same point on the retina regardless of eye movement.

In order to accomplish the closed loop tracking of the eye motion, image processing module 42 includes an image processing unit 50 using image analysis software such as described in US Patent Publication 2011/0142370; 2011/0141300; and 2011/0141226. Image matching is also shown in U.S. Pat. No. 7,961,982. Note, U.S. Pat. No. 7,925,051 measures local motion between successive images.

Most importantly, in one embodiment the method described in the The Dual Bootstrap Iterative Closest Point Algorithm with Application to Retinal Image Registration of the Stewart el at reference mentioned above may be used to capture all of the information in the sensed region and using feature extraction provide an artifact free motion vector that describes the displacement of the region due to eye movement.

With such image analysis, module 42 measures the position of a reference region on the image from auxiliary imager 36 for a given frame and then measures the position of this reference region on a subsequent frame using registration algorithms. The shifts in the features tracked in these regions in for instance the X and Y directions are utilized to calculate the movement of the region in the image from a point P₁ to a point P₂ as illustrated at 52. This movement is captured as a mapping vector. These mapping vectors are then utilized to derive matrix 44 which in turn can be utilized to derive scan mirror pattern offsets. The matrix is applied to drive 46 to generate the corresponding drive signals to offset the scan mirror patterns. These drive signals are applied to drive actuators 22 and 24 to offset whatever rotation is initially provided by these actuators to provide the OCT scan, thus to cancel the effect of eye motion.

What is now described is the operation of the matrix and drive for the scan mirror axes.

Note, the complexity of the matrix calculations and how they are derived and used to create the scan pattern offsets can vary greatly. In its simplest form, the matrix can be used to describe a simple rigid geometric transform known as an Isometry Transformation, which is basically to cut from the reference image and overlay onto the subsequent frame. In an isometry transformation, there are 3 degrees of freedom (DOF), 2 associated with Translation (left to right, up and down) and 1 associated with rotation.

This can be represented by the following linear transformation matrix:

$$( \begin{matrix}X 2 \\ Y 2 \\ 1\end{matrix} ) = ( \begin{matrix}a & c & tx \\ b & d & ty \\ u & v & w\end{matrix} ) * ( \begin{matrix}X 1 \\ Y 1 \\ 1\end{matrix} )$$

Where X2,Y2 is a point on the transformed image (or current frame), and X1,Y1 is a point on the source image (or reference frame). For a simple isometry transformation, one can utilize tx and ty to characterize translation in the X and Y direction, and rotation of an angle θ using a=cos θ, b=sin θ, c=−sin θ, d=cos θ (u, v and w are static positions and would be 0, 0, 1 respectively).

By expanding the input values of the matrix, one can implement further levels of complexity to achieve a Similarity Transformation involving 4 DOF, or an Affine Transformation, which is a linear transformation with 6 DOF, by implementing Translation, Skew (shearing in the X or Y dimension), Scale (minification or magnification in the X or Y dimension). This can be further expanded to include Perspective Translation, and with Quadratic Transformation one can reach 12 degrees of freedom and achieve a sub-pixel accuracy exceeding the resolution of the auxiliary imager. It should also be noted that these matrices can be further expanded still, to include a 3 dimensional dataset registration as described in the anterior segment OCT.

Using these matrices and available information from the images, one can translate scan points from the desired location on the original reference image, to the corresponding location on the current frame from the auxiliary imager.

Thus if one wants to go from a point on the surface image, one inputs the values of X1 and Y1. Then one inputs the matrix values derived from the registration software. Thereafter the matrix multiplication results in the equivalent scan point on the new image. Note that the offset of the scanner is the differences between the old and new points.

With regard to scan skipping, the problem is that since one is using an image processing based technique one is operating much slower than an electro-optical technique which is the problem U.S. Pat. No. 7,805,009 attempts to addresses. This patent attempts to address the slowness of the process by analyzing the changes in position based on a line of data so that the system can process data very quickly as the lines are coming in. The problem with this approach is that one needs a more data rich region in which the whole image is captured so that the image can be completely processed. This adds to a slower processing rate.

To solve this problem in the subject invention, the basic theory is that the eye is stable for small periods of time and then large or rapid involuntary movements occur so that what one tries to do is to discriminate those large movements and reset the scan process after the motions have occurred and have settled down, after which there is a period of stability. One can then continually scan in this way so that one is discarding the data during the large motions while accepting the data during slow eye movement periods.

The first step in applying this approach is to develop a motion threshold 60 applied to an A-scan skipping module 62 that specifies that anything below the motion threshold is going to be a very minor and negligible motion, the data from which can be accepted. Anything above the motion threshold is large scale motion or rapid motion that is ignored. So by applying the threshold, if the threshold gets exceeded, one ignores the data that is coming out from that frame and from the previous frame. One then continues ignoring the data until the matrix indicates that the motion is now below the threshold.

As will be appreciated, the matrix values are developed from the results of the algorithm that detects the change in region position.

In one scenario where one is operating at 30 frames per second, a majority of those frames within one second, for instance 15, will come out with relatively little motion and are accepted. Then at the period of time at which a large jump occurs, the data is ignored. Thereafter the retina will then slow down again and stabilize. As a result one simply does not throw out all of the data, but only the data during that small period of time when the fast motion occurs, i.e. 5 or 6 frames, after which one resets the scan pattern, with the scan going back to the last known point that occurs prior to the rapid motion, which in this example would be frame 15. One then resumes scanning the same points again from frame 15, basically the points that were acquired during the motion of the eye.

While the present invention has been described in connection with the preferred embodiments of the various figures, it is to be understood that other similar embodiments may be used or modifications or additions may be made to the described embodiment for performing the same function of the present invention without deviating therefrom. Therefore, the present invention should not be limited to any single embodiment, but rather construed in breadth and scope in accordance with the recitation of the appended claims.

What is claimed is:

1. A method for eliminating the effects of the movement of the eye on an optical coherence tomography engine which scans a portion of the eye utilizing a scan module, comprising the steps of:
detecting motion of the eye from an image of the eye generated by an auxiliary imager having a predetermined frame rate by comparing a data rich reference region at a point in one image taken from a frame to the position of a corresponding region in the image associated with another frame, with the displacement in region position sensing the motion of the eye; and,
utilizing the sensed motion of the eye in terms of region position displacement between frames to generate offsets for the scan module in the optical coherence tomography scanner to counter sensed eye movement.

2. The method of claim 1, wherein said optical coherence tomography engine includes one of a spectral domain, frequency domain, Fourier domain and time domain optical coherence tomography scanner.

3. The method of claim 1, wherein the scan module includes at least one scanning mirror.

4. The method of claim 1, wherein the step of detecting eye motion includes the step of generating an en face surface view of the eye as processed from optical coherence tomography scan data.

5. The method of claim 1, wherein the step of generating the offsets for the scan module includes generating a matrix that is used to calculate scan offsets from the detected region displacement.

6. The method of claim 5, wherein said matrix is used to register and align the optical coherence tomography scan depth vectors in a three dimensional space.

7. The method of claim 1, wherein the auxiliary imager includes a line scan camera associated with a line scan ophthalmoscope.

8. The method of claim 1, wherein the auxiliary imager includes a point scan detector associated with a scanning laser ophthalmoscope.

9. The method of claim 1, wherein the auxiliary imager includes a high speed line scan ophthalmoscope.

10. The method of claim 1, wherein the auxiliary imager includes a scanning laser ophthalmoscope.

11. The method of claim 1, wherein the image from the auxiliary imager is displayed on a computer monitor.

12. The method of claim 11, wherein a subsequent optical coherence tomography scan registered to an image of the eye containing the reference region is displayed on the monitor and wherein the displacement between the subsequently generated image of the region and the originally-generated image of the region is canceled by adjusting the scan module such that optical coherence tomography scans impinge on the same region of the eye.

13. The method of claim 1, wherein the predetermined frame rate is equal to or greater than 5 frames per second.

14. The method of claim 1, and further including the step of using scan skipping to ignore data from sensed eye motions above a predetermined eye motion threshold and causing the ignored data to be rescanned.

15. The method of claim 1, wherein the portion of the eye scanned includes the retina of the eye.

16. The method of claim 1, wherein the portion of the eye scanned includes the posterior portion of the eye.

17. The method of claim 1, wherein the portion of the eye scanned includes the anterior portion of the eye.

18. The method of claim 1, wherein the auxiliary image is analyzed to detect Purkinje images so as to obtain directional vectors of the eye's gaze.

19. The method of claim 1, wherein the portion of the eye scanned includes the iris.

20. The method of claim 1, wherein the initial reference image is taken from a previous auxiliary image of the same eye, thereby causing the currently scanned region to be coincident with the previously scanned region, thus creating a comparison scan over time.

21. The method of claim 1, wherein said reference region is adapted to be specified by a user and its location is adapted to be specified by referencing an auxiliary image of the eye.

22. Apparatus for eliminating the effect of eye movement on the output of an optical coherence tomography scanner comprising:
an optical coherence tomography engine having a scan module, said optical coherence tomography engine including an auxiliary imager, said image created from scanning a portion of the eye at a predetermined frame rate and providing as an output therefrom an image of the scanned portion of the eye, said scanned portion of the eye including a data rich region;
an image processing unit for determining motion of the eye by tracking a change in position of a data rich reference region in the image produced by said auxiliary imager from one frame to another, said image processing unit including a calculator for calculating the change in position of said reference region from one frame to another and for calculating scan module offsets from the calculated change in position of said reference region; and,
a feedback loop coupled to said image processing unit for offsetting said optical coherence tomography scan module to counter the sensed motion of the eye as determined by said image processing unit.

23. The apparatus of claim 22, wherein said scan module has an optical axis and wherein said auxiliary imager has an optical axis aligned with the optical axis established by the scan mechanism.

24. The apparatus of claim 22, wherein said method of creating an auxiliary image includes a high speed line scan ophthalmoscope.

25. The apparatus of claim 22 wherein said auxiliary imager generates an en face surface view of the eye as processed from optical coherence tomography scan data.

26. The apparatus of claim 22, wherein said scan module includes one or more scanning mirrors.

27. The apparatus of claim 22, wherein said auxiliary imager includes a high speed scanning laser ophthalmoscope.

28. The apparatus of claim 22, wherein said optical coherence tomography engine includes one of a spectral domain scanner, a frequency domain scanner, a Fourier domain scanner and a time domain scanner.

29. The apparatus of claim 22, wherein the portion of the eye scanned by said optical coherence tomography engine includes the anterior portion of the eye.

30. The apparatus of claim 22, wherein the portion of the eye scanned by said optical coherence tomography engine includes a posterior portion of said eye.

31. The apparatus of claim 30, wherein said posterior portion of the eye includes the retina.

32. The apparatus of claim 22, wherein said optical coherence tomography engine produces a display of the depth profile of the tissue of the eye.

33. The apparatus of claim 32, wherein said optical coherence tomography engine produces an A scan that detects the depth profile of the tissue of the eye.

34. The apparatus of claim 33, wherein said optical coherence tomography engine produces a B scan of the eye along a scan line so as to provide a two dimensional rendition of the depth profile of the scanned tissue forming a slice along a predetermined scan line.

35. The apparatus of claim 22, and further including a scan skipping module operably coupled to said image processing unit for ignoring rapid eye movement when said eye movement exceeds a predetermined motion threshold indicative of rapid eye movement, thus to ignore the data in a scan due to said rapid eye movement, said scan skipping causing the ignored data to be rescanned.

36. The apparatus of claim 22, wherein said reference region is selected from a region of a previous auxiliary image of the same eye, thereby causing the current scan to be coincident with the previous scan, thus creating a comparison scan over time.

37. The apparatus of claim 22, wherein said reference region and its location on the eye are adapted to be defined by a user, wherein said optical coherence tomography scanner has a scan pattern which scans said defined reference region.
